(19) United States
(12) Patent Application Publication
(10) Pub. No.: US 2011/0275869 A1
(43) Pub. Date: Nov. 10, 2011

(54) PROCESS FOR PRODUCING SYNTHESIS GAS AND AT LEAST ONE ORGANIC LIQUID OR LIQUEFIABLE MATERIAL OF VALUE

(75) Inventors: ROMAN PROCHAZKA, MANNHEIM (DE); STEFAN BITTERLICH, DIRMSTEIN (DE); OTTO MACHHAMMER, MANNHEIM (DE); STEPHAN DEUERLEIN, LUDWIGSHAFEN (DE); DIRK KLINGLER, MANNHEIM (DE); EMMANOUIL PANTOUFLAS, MANNHEIM (DE); ALOIS KINDLER, GRUENSTADT (DE); BERND ZOELS, OBRIGHEIM (DE)

(73) Assignee: BASF SE, LUDWIGSHAFEN (DE)

(21) Appl. No.: 13/101,368

(22) Filed: May 5, 2011

Related U.S. Application Data
(60) Provisional application No. 61/332,201, filed on May 7, 2010.

(51) Int. Cl. C07C 1/00; C07C 39/00; C01B 3/34

(52) U.S. Cl. 585/242; 252/373; 585/240; 568/716; 568/700

(57) ABSTRACT

The present invention relates to a process for producing synthesis gas and at least one organic liquid or liquefiable material of value, wherein
a) a biomass starting material is provided,
b) the biomass starting material is subjected to a decomposition,
c) at least one aromatics-enriched fraction C1) and at least one aromatics-depleted fraction C2) are optionally isolated from the decomposed material obtained in step b),
d) the decomposition product from step b) or the aromatics-enriched fraction C1) from step c) is fed into a dealkylation zone and reacted in the presence of hydrogen and/or water vapor,
e) a discharge is taken from the dealkylation zone and subjected to a separation to give at least one organic liquid or liquefiable material of value and at least one stream enriched in components which are more volatile than the organic material of value,
f) the stream enriched in components which are more volatile than the organic material of value which is obtained in step e) is at least partly used for producing synthesis gas.

### BACKGROUND OF THE INVENTION

The present invention relates to a process for producing synthesis gas and at least one organic liquid or liquefiable material of value.

The large amounts of biomass produced continually by nature have hitherto been used to only a small extent as renewable raw material for use as material or for the generation of energy. To conserve resources of raw materials, processes which allow the replacement of fossil raw materials by biomass starting materials are required. In order to achieve high efficiency, attempts are made to use as much of the biomass material provided as possible.

Aromatic compounds having a low molecular weight and especially phenolic compounds have found wide use as intermediates and products of value. They serve, for example, as precursor for various resins, surface-active compounds, specialty chemicals, etc. It is known that such compounds can be prepared from biomass materials and specifically from lignin-comprising starting materials. However, there is a further need for a simple, inexpensive process which allows the preparation of a variety of aromatic products for various fields of use. It is advantageous for further materials of value to be able to be obtained in addition to the desired aromatic products and if possible be used again in the process for aromatics production or a process coupled therewith, e.g. a process for pulp production. Such a material of value, which is used in major industrial processes, is known as “synthesis gas”, a gas mixture comprising carbon monoxide and hydrogen. Areas in which synthesis gas is used include, for example, hydrogenation, hydroformylation, carbonylation, methanol synthesis, synthesis of hydrocarbons by the Fischer-Tropsch process, etc.

Subjecting streams from various digestion processes of lignin- or lignocellulose-comprising materials to an after-treatment to isolate materials of value is known.

U.S. Pat. No. 2,057,117 describes a process for preparing vanillin, in which a starting material selected from lignocellulose, a crude lignin extract and ligninsulfonic acid is heated with an aqueous alkali metal hydroxide solution under superatmospheric pressure and the reaction mixture obtained is admixed with sulfuric acid in order to precipitate organic constituents and convert the vanillin into a soluble form.

WO 99/10450 describes a process for converting lignin into a hydrocarbon fuel. Here, lignin is subjected to a base-catalyzed depolymerization and subsequently hydroprocessing. This hydroprocessing comprises a hydrodeoxygenation and mild hydrocracking. The latter is carried out under conditions under which partial hydrogenation of the aromatic rings occurs.

WO 2008/027699 A2 describes a process in which lignin originating from a pyrolysis of biomass is, after water-soluble constituents have been separated off, decarboxylated and hydrodeoxygenated and the organic products from this process step are subsequently subjected to hydrocracking.

WO 2010/026244 describes an integrated process for producing pulp and at least one low molecular weight material of value, in which
a) a lignocellulose-comprising starting material is provided and subjected to digestion with a treatment medium,
b) a cellulose-enriched fraction and at least one cellulose-depleted fraction are isolated from the digested material, where the cellulose-depleted fraction comprises at least part of the treatment medium from step a),
c) the cellulose-depleted fraction is subjected to a treatment to give at least one low molecular weight material of value, and
d) the material/materials of value is/are isolated from the treatment product obtained in step c).

In an embodiment of the process, a cellulose-enriched fraction and a lignin-enriched fraction are isolated from the digested material, the lignin-enriched fraction is subjected to a depolymerization and an aromatics composition is isolated from the depolymerization product.

WO 2009/108601 describes a process for producing a starting material for biorefinery processes for producing a biofuel from a lignin-comprising starting material. Here, lignin from a black liquor of the pulping process or else the black liquor itself is subjected to hydroprocessing in the presence of a hydrogen-comprising gas and a catalyst on an amorphous or crystalline oxidic support. Specifically, a heterogeneous molybdenum sulfide catalyst is used. When black liquor is used, the hydroprocessing can also be carried out in two stages. The process can either be carried out at a refinery site to which the lignin or black liquor is transported or directly on the site of a paper mill. The biorefinery process following hydroprocessing is not described in more detail.

WO 2009/108599 has a disclosure content comparable to WO 2009/108601, with the focus on paper production.

In Angew. Chem. 2008, 120, 9340-9351, M. Stöcker describes the catalytic conversion of lignocellulose-rich biomass to produce BTL (biomass-to-liquid) fuels in biorefineries. The use of a lignin material obtained from the biomass in a pyrolysis to produce biooil and a further work-up to give phenolic resins, synthesis gas, etc., is also shown schematically.

US 2009/0227823 describes a process for producing at least one liquid hydrocarbon product from a solid hydrocarbon starting material (e.g. a lignocellulose material), in which the starting material is subjected to catalytic pyrolysis and the pyrolysis products are subjected to a catalyzed after-reaction to give liquid products.

In Chem. Rev. 2006, 106, 4044-4098, G. W. Huber et al. describe synthesis of fuels from biomass. According to that article, lignocellulose materials can in principle be converted into liquid fuels by means of three routes which differ in their primary step: gasification of synthesis gas, pyrolysis to biooil, hydrolysis to give sugars and lignin. The biooils obtained in the pyrolysis can subsequently be subjected to hydrodeoxygenation in the presence of hydrogen or to steam reforming.

### BRIEF SUMMARY OF THE INVENTION

It has surprisingly been found that the combination of decomposition and dealkylation allows firstly organic liquid or liquefiable materials of value (especially single-ring aromatics which are unalkylated or have a low degree of alkylation) and secondly synthesis gas as further product of value to be produced from a large number of biomass materials. The synthesis gas obtained in this way can advantageously be used in the process of the invention itself or a nearby process coupled therewith, e.g. a process for producing pulp.

The invention firstly provides a process for producing synthesis gas and at least one organic liquid or liquefiable material of value, wherein
a) a biomass starting material is provided,
b) the biomass starting material is subjected to a decomposition,
c) the decomposed material obtained in step b) is optionally separated into at least one aromatics-enriched fraction C1) and at least one aromatics-depleted fraction C2),
d) the decomposition product from step b) or the aromatics-enriched fraction C1) from step c) is fed into a dealkylation zone and reacted in the presence of hydrogen and/or water vapor,
e) a discharge is taken from the dealkylation zone and subjected to a separation to give at least one organic liquid or liquefiable material of value and at least one stream enriched in components which are more volatile than the organic material of value,
f) the stream enriched in components which are more volatile than the organic material of value which is obtained in step e) is at least partly used for producing synthesis gas.

### BRIEF DESCRIPTION OF THE SEVERAL VIEWS OF THE DRAWINGS

FIG. 1 shows a preferred embodiment of the process of the invention.

FIG. 2 shows a vaporization of an aromatics-comprising stream as is obtained in the separation by absorption and distillation of the discharge from the dealkylation zone.

### DETAILED DESCRIPTION OF THE INVENTION

For the purposes of the present patent application, the term “biomass” refers to a plant material of nonfossil origin. Biomass also includes plants and plant parts which have died off, for example dead wood, straw, leaves, etc. The term biomass also comprises products obtained by subjecting a plant material of nonfossil origin to a chemical and/or physical treatment. Such products include, in particular, the products from the digestion and the fractionation of lignocellulose, e.g. lignin. Coal, petroleum, natural gas, peat and their upgrading products, e.g. coke, in particular, do not count as biomass.

For the purposes of the invention, the term “synthesis gas” refers to a gas mixture comprising carbon monoxide and hydrogen. This gas mixture can additionally comprise further gases such as CO₂, CH₄, etc. The process of the invention advantageously makes it possible to produce synthesis gas having a high content of carbon monoxide and hydrogen.

For the purposes of the present invention, an organic liquid or liquefiable material of value is an organic compound or a composition comprising at least two organic compounds which are liquid or can be liquefied without decomposition under standard conditions (20° C., 1013 mbar). Here, liquefaction refers to the transition from the solid state into the liquid state in the sense of melting and not solubilization with addition of a solvent.

The organic material of value is, for example, selected from unfunctionalized and functionalized aliphatic, cycloaliphatic and aromatic hydrocarbons. These include especially alkanes, (e.g. pentane, hexane, etc.), alkenes, alkadienes, alkanols (e.g. methanol, ethanol, etc.), aliphatic aldehydes (e.g. acetaldehyde, etc.), cycloalkanes, cycloalkenes, cycloalkadienes, cycloalkanols, cycloalkadienols, cycloalkane polyols having more than two OH groups and unfunctionalized and functionalized aromatic hydrocarbons.

The organic liquid or liquefiable material of value is preferably selected from unfunctionalized and functionalized aromatics. Functionalized aromatics preferably have at least one substituent selected from C₁-C₄-alkyl, OH, C₁-C₄-alkoxy, formyl, C₁-C₄-acyl, and combinations thereof. The organic material of value is in particular selected from benzene, alkylated benzenes (e.g. toluene and xylenes), relatively highly condensed aromatic hydrocarbons, monoalkylated, dialkylated and polyalkylated, relatively highly condensed aromatics, phenol, monoalkylated, dialkylated and polyalkylated phenols, relatively highly condensed aromatics having one, two or more than two OH groups, monoalkylated, dialkylated and higher-alkylated, relatively highly condensed aromatics having one, two or more than two OH groups, alkoxylated derivatives of the abovementioned aromatic alcohols and mixtures thereof.

In a specific embodiment, the organic liquid or liquefiable material of value prepared according to the invention is an aromatics composition having a high content of single-ring aromatics which are unalkylated or have a low degree of alkylation. For the purposes of the present invention, an aromatics composition having a high content of single-ring aromatics which are unalkylated or have a low degree of alkylation is a composition which, based on its total weight, comprises at least 50% by weight of single-ring aromatics. The total content of unalkylated, unalkoxylated, at most monohydroxylated and monoalkylated aromatics is, based on the total weight of the aromatics composition, at least 50% by weight.

For the purposes of the invention, “dealkylation” refers to a reaction of the substituted and/or polycyclic aromatic compounds comprised in an aromatics composition in the presence of hydrogen and/or water vapor, with these being at least partly transformed in such a way that substituents are replaced by hydrogen and/or compounds comprising a plurality of aromatic rings are cleaved to give compounds having a lower number of rings. The substituents replaced by hydrogen are selected from alkyl groups, hydroxy groups, alkoxy groups, aryloxy groups, etc. For the purposes of the present invention, the term “dealkylation” also refers to reactions different therefrom which are associated with a decrease in the molecular weight, e.g. dehydroxylation, dealkoxylation or aromatics cleavage. The term aromatics cleavage refers to a reaction in which essentially the number of aromatic rings per molecule is reduced without the aromatic rings themselves being destroyed.

### Provision of a Biomass Starting Material (Step a)

A lignin-comprising material is preferably provided as biomass starting material in step a) of the process of the invention.

Suitable lignin-containing starting materials are pure lignin and lignin-comprising compositions. The lignin content of the compositions is not critical within a wide range; it is merely the case that at lignin contents which are too low, the process can no longer be operated economically.

A lignin-comprising starting material which comprises at least 10% by weight, preferably at least 15% by weight, based on the dry mass of the material, of lignin is preferably provided in step a). Preference is given to lignin-comprising compositions which comprise from 10 to 100% by weight, particularly preferably from 15 to 95% by weight, based on the dry mass of the material, of lignin. For the purposes of the present invention, the term dry mass is defined as in the standard ISO 11465.

Lignocellulose-comprising materials are also suitable for providing a lignin-comprising starting material for use in the process of the invention. Lignocellulose forms the structural framework of the cell walls of plants and comprises lignin, hemicelluloses and cellulose as main constituents. Further constituents of the cell walls of plants and thus of the lignocellulose-comprising materials obtained therefrom are, for example, silicates, extractable low molecular weight organic compounds (known as extractables, e.g. terpenes, resins, fats), polymers such as proteins, nucleic acids and vegetable gum (known as exudate), etc.

Lignin is a biopolymer whose basic unit is essentially phenylpropane which, depending on the natural source, may be substituted by one or more methoxy groups on the phenyl rings and by a hydroxy group on the propyl units. Typical structural units of lignin are therefore p-hydroxyphenylpropane, guaiacylpropane and syringylpropane which join to one another by ether bonds and carbon-carbon bonds.

Both lignocellulose-comprising materials which are used in the natural composition without further chemical treatment, e.g. wood or straw, and lignin-comprising streams from the processing of lignocellulose, e.g. from processes for producing cellulose (pulp processes), are suitable as biomass starting material for the process of the invention.

The lignocellulose materials which can be used according to the invention can be obtained, for example, from wood fibers and plant fibers as starting material. Preferred lignocellulose materials are those from wood and residues of the wood processing industry. They include, for example, the various types of wood, e.g. broadleaved tree wood such as maple, birch, pear tree, oak, alder, ash, eucalyptus, common beech, cherry tree, lime, nut tree, poplar, willow, etc. and conifer timbers such as Douglas fir, spruce, yew, hemlock, pine, larch, fir, cedar, etc. Wood can be divided not only into wood from broadleaved trees and that from conifers but also into “hardwoods” and “softwoods”, which is not synonymous with the terms broadleaved tree timbers and conifer timbers. In contrast to hardwood, the term softwood refers to lighter wood (i.e. wood having a dried density below 0.55 g/cm³, for example willows, poplars, limes and virtually all conifers). All hardwoods and all softwoods are in principle suitable for use in the process of the invention. The wood used in the process of the invention can also be present in manufactured form, e.g. in the form of pellets. Suitable residues from the wood-processing industry are not only scrap wood but also sawdust, parquetry grinding dust, etc. Further suitable lignocellulose materials are natural fibers such as flax, hemp, sisal, jute, straw, coconut fibers, switchgrass (Panicum virgatum) and other natural fibers. Suitable lignocellulose materials are also obtained as residues in agriculture, e.g. in the harvesting of cereal (wheat straw, maize straw, etc.), maize, sugarcane (bagasse), etc. Suitable lignocellulose materials are also obtained as residue in forestry, e.g. in the form of branches, bark, wood chips, etc. Another good source of lignocellulose materials is short rotation crops which allow high biomass production on a relatively small area.

In step a), preference is given to providing a lignin-comprising stream from the digestion of a lignocellulose material for producing cellulose (pulp), preferably a black liquor, in particular a black liquor from the kraft digestion (sulfate digestion), as biomass starting material.

In a preferred embodiment, a lignocellulose-comprising material is subjected to digestion and a cellulose-enriched fraction and a lignin-enriched (and at the same time cellulose-depleted) fraction are isolated from the digested material in order to provide the biomass starting material. The lignin-enriched fraction then serves, optionally after a further work-up, as biomass starting material for the process of the invention. In this embodiment, a lignocellulose-comprising material is thus subjected to a first decomposition in step a) of the process of the invention, a lignin-enriched material is isolated therefrom and the latter is subsequently subjected to a second decomposition in step b).

Processes for digesting lignocellulose-comprising materials to produce cellulose are known in principle. In principle, lignin-comprising streams from all digestion processes known to those skilled in the art are suitable for use as biomass starting material. These processes can basically be divided into aqueous-alkaline processes, aqueous-acidic processes and organic processes on the basis of the treatment medium used. An overview of these processes and the digestion conditions may be found, for example, in WO 2010/026244.

The treatment medium used for digesting the lignocellulose-comprising materials is capable of solubilizing at least part of the lignin. The cellulose comprised in the lignocellulose-comprising material, on the other hand, is generally not solubilized or solubilized to only a small extent in the treatment medium. The isolation of a cellulose-enriched fraction is then preferably carried out by filtration or centrifugation.

A lignin-comprising (cellulose-depleted) fraction which in addition to lignin comprises at least one further component selected, for example, from hemicellulose, cellulose, degradation products of the abovementioned components, digestion chemicals and mixtures thereof is preferably isolated from the digested material.

In many cases, it is not critical to the decomposition in step b) if a lignin-comprising starting material which comprises at least one further component in addition to lignin is used as biomass starting material.

If a lignin-comprising fraction which comprises at least one further component in addition to lignin is used for providing the lignin-comprising starting material, then at least part of the compounds other than lignin can be removed before the decomposition in step b). The components removed from the lignin-comprising fraction (organic components and/or inorganic process chemicals) are preferably passed to a further work-up and/or thermal utilization, preferably in the process for cellulose production from which the lignin-comprising fraction was obtained.

To remove at least part of the compounds other than lignin, the pH of the lignin-comprising fraction can firstly be set to a suitable value. Lignin-comprising fractions from aqueous-alkaline processes (e.g. the kraft process) can be admixed with an acid to adjust the pH. Suitable acids are, for example, CO₂, mineral acids such as hydrochloric acid, sulfuric acid and phosphoric acid. Particular preference is given to CO₂ (or the carbonic acid resulting therefrom by reaction with water) as acid. Preference is given to using CO₂ from an offgas stream from the process of the invention or a pulp process coupled with the process of the invention. For example, the offgas from a black liquor combustion (recovery boiler) or a lime kiln is suitable. Here, the offgas can be introduced either directly or after removal of the other components (e.g. by means of a scrubbing process such as a Benfield scrub) into the lignin-comprising fraction. The carbonates and/or hydrogencarbonates formed as a result of the addition of CO₂ can generally be easily recirculated to the coupled pulp process, e.g. into a black liquor taken off beforehand for isolating lignin. The use of CO₂ for adjusting the pH of the lignin-comprising fraction is thus associated with lower costs than the use of other acids and also generally allows good integration into a pulp process.

Lignin-comprising fractions from aqueous-acidic processes can be admixed with a base to adjust the pH. Suitable bases are, for example, alkali metal bases such as sodium hydroxide or potassium hydroxide, alkali metal carbonates such as sodium carbonate or potassium carbonate, alkali metal hydrogencarbonates such as sodium hydrogencarbonate or potassium hydrogencarbonate and alkaline earth metal bases such as calcium hydroxide, calcium oxide, magnesium hydroxide or magnesium carbonate, and also ammonia or amines.

In step a), the removal of at least part of the compounds other than lignin from the lignin-comprising fraction is preferably effected by filtration, centrifugation, extraction, precipitation, distillation, stripping or a combination thereof. A person skilled in the art will be able to control the composition of the lignin-comprising fraction and thus of the lignin-comprising starting material for the decomposition in step b) via the isolation method. The at least partial separation of the components other than lignin can be effected in one or more stages. Customary filtration processes are, for example, cake filtration and deep bed filtration (e.g. as described in A. Rushton, A. S. Ward, R. G. Holdich: Solid-Liquid Filtration and Separation Technology, VCH Verlagsgesellschaft, Weinheim 1996, pages 177ff., K. J. Ives, in A. Rushton (editor): Mathematical Models and Design Methods in Solid-Liquid Separation, NATO ASI Series E No. 88, Martinus Nijhoff, Dordrecht 1985, pages 90ff.) and crossflow filtrations (e.g. as described in J. Altmann, S. Ripperger, J. Membrane Sci. 124 (1997), pages 119-128). Customary centrifugation processes are described, for example, in G. Hultsch, H. Wilkesmann, “Filtering Centrifuges,” in D. B. Purchas, Solid-Liquid Separation, Upland Press, Croydon 1977, pages 493-559; and in H. Trawinski, Die äquivalente Klärfläche von Zentrifugen, Chem. Ztg. 83 (1959), pages 606-612. Extraction can be carried out using, for example, a solvent which is not miscible with the treatment medium from pulp production or at least one solvent which has a miscibility gap and in which lignin and optionally further desired components are soluble in a sufficient amount. The removal of components which can be vaporized without decomposition from the lignin-comprising fraction can be carried out by customary distillation processes known to those skilled in the art. Suitable apparatuses for the work-up by distillation comprise distillation columns such as tray columns equipped with bubble caps, sieve plates, sieve trays, ordered packing, random packing elements, valves, side offlakes, etc., evaporators such as thin film evaporators, falling film evaporators, forced circulation evaporators, Sambay evaporators, etc., and combinations thereof.

In a specific embodiment, a lignin-comprising stream which is derived from the digestion of a lignocellulose material and still comprises at least part of the liquid treatment medium from the digestion is used to provide the lignin-comprising starting material in step a). The lignin-comprising stream is then preferably subjected to precipitation of a lignin-comprising fraction, followed by partial or complete removal of the liquid components, to provide the lignin-comprising starting material for the decomposition in step b).

The lignin-comprising starting material is preferably provided in a process for producing cellulose (pulp) into which the production according to the invention of synthesis gas and at least one organic liquid or liquefiable material of value is integrated.

In a specific embodiment, the removal of at least part of the liquid compounds is then carried out during the process for producing pulp. Thus, for example, a black liquor taken off before or during the course of the individual evaporation steps of the parent pulp process can be used for the provision of the lignin-comprising starting material.

A lignin-comprising stream from the digestion of a lignocellulose material by means of an alkaline treatment medium is preferably used to provide the biomass starting material in step a). Particular preference is given to using a black liquor, in particular a black liquor from the sulfate digestion (kraft digestion). To provide a lignin-comprising material, a black liquor from the kraft digestion can firstly be acidified to precipitate at least part of the lignin comprised and the precipitated lignin can subsequently be isolated. The abovementioned acids are suitable for acidification. In particular, CO₂ is used. The pH of the black liquor is preferably reduced to a value of not more than 10.5. The isolation of the precipitated lignin is preferably effected by a filtration process. Suitable filtration processes are those mentioned above. If desired, the lignin isolated can be subjected to at least one further work-up step. Such steps include, for example, a further purification, preferably washing with a suitable washing medium. Suitable washing media are, for example, mineral acids such as sulfuric acid, preferably in aqueous solution. In a specific embodiment, a lignin-comprising material can then be provided by firstly acidifying a black liquor from the kraft digestion with CO₂ to precipitate at least part of the lignin comprised, subsequently isolating the precipitated lignin by filtration and subjecting the filtrate to a scrub with sulfuric acid.

A process for isolating lignin from a black liquor by precipitation using CO₂ is described in WO 2008/079072, which is hereby incorporated by reference. The Lignoboost process described in WO 2006/038863 (EP 1797236 A1) and WO 2006/031175 (EP 1794363 A1), which are likewise incorporated by reference, is also particularly suitable.

### Decomposition (Step b)

In step b) of the process of the invention, the biomass starting material is subjected to a decomposition to give a decomposition product comprising components whose average molecular weight is significantly below the average molecular weight of the components comprised in the biomass starting material.

In a specific embodiment, a lignin-comprising starting material is used for the decomposition in step b). According to this embodiment, the decomposition product obtained in step b) preferably comprises predominantly components having a molecular weight of not more than 500 g/mol, particularly preferably not more than 400 g/mol, in particular not more than 300 g/mol.

The decomposition in step b) can in principle be carried out according to two variants, which are described in detail below. The first variant comprises a pyrolysis and correspondingly leads to a pyrolysis product. The second variant comprises a reaction in the presence of a liquid decomposition medium and accordingly leads to a product of the liquid decomposition.

### 1st Variant: Pyrolysis

In a first variant of the process of the invention, the biomass starting material, especially the lignin-comprising starting material, is subjected to a pyrolysis in step b). For the purposes of the invention, “pyrolysis” is a thermal treatment of the biomass starting material, with molecular oxygen not being introduced or introduced only in a small amount. Here, a small amount is an amount which is significantly smaller than the amount necessary for complete oxidation of the carbon comprised in the biomass starting material to CO₂. The amount of molecular oxygen introduced in the pyrolysis is preferably at least 50 mol % below, particularly preferably at least 75 mol % below, in particular at least 90 mol % below, the amount necessary for complete oxidation of the carbon comprised in the biomass starting material to CO₂. The pyrolysis generally occurs endothermically. In this variant of the process of the invention, the decomposition product is at least partly obtained in gaseous form.

The pyrolysis can be carried out batchwise or continuously. Continuous pyrolysis is preferred.

The pyrolysis is carried out in at least one pyrolysis zone. The biomass starting material, especially the lignin-comprising starting material, can be introduced into a pyrolysis zone by means of suitable transport devices, e.g. screw conveyors or pneumatic transport.

To carry out the pyrolysis, the biomass starting material, especially the lignin-comprising starting material, is preferably used in predominantly solid form. For the purposes of the invention, “predominantly solid form” means that the starting material used for the pyrolysis has, under normal conditions (20° C., 1013 mbar), a liquid content of not more than 70% by weight, particularly preferably of not more than 50% by weight, based on the total weight of the starting material. For the pyrolysis, the biomass starting material, especially the lignin-comprising starting material, is then used as, for example, a moist or predried solid.

The pyrolysis zone can have various embodiments, e.g. as rotary tube furnace or fluidized bed. Both stationary and circulating fluidized beds are suitable. In the embodiment of the pyrolysis zone as a fluidized bed, a fluidizing gas (preferably steam or a gas mixture from one of the subsequent process steps) and as fluidized material a particulate material which is inert under the prevailing conditions are introduced. A particularly suitable inert material is silica sand. Such a fluidized-bed process is described, for example, in U.S. Pat. No. 4,409,416 A. In an alternative embodiment, the pyrolysis zone comprises at least one fixed bed. The fixed beds can comprise at least one inert fixed bed and/or at least one catalytically active fixed bed. If the process of the invention is operated using at least one fixed bed as pyrolysis zone, operation in cycles, where a pyrolysis phase is followed by a burning-off phase in order to remove relatively nonvolatile components from the fixed bed, can be advantageous.

For the pyrolysis, a fluidizing gas can be introduced into the pyrolysis zone. Preferred fluidizing gases are steam, carbon dioxide, nitrogen, etc. or mixtures of these gases.

In a first preferred embodiment, the pyrolysis is not carried out with addition of hydrogen. In this embodiment, the hydrogenating reaction occurs essentially in the dealkylation step d).

In a second preferred embodiment, the pyrolysis is carried out with addition of hydrogen. This embodiment of the pyrolysis can also be referred to as hydrocracking. In hydrocracking, the biomass starting material, especially lignin, is cleaved into low molecular weight fragments by action of hydrogen. The pyrolysis with addition of hydrogen is preferably carried out in suspension. Furthermore, it is preferably carried out using a catalyst and/or under high pressure. Such a process is described, for example, in U.S. Pat. No. 4,420,644 and in H. L. Churn et al., Adv. Solar Energy, Vol. 4 (1988), 91 ff.

In a further preferred embodiment, an evaporated black liquor from the kraft process is used for the pyrolysis. Such a process is described, for example, in U.S. Pat. No. 3,375,283. The black liquor is in this case present predominantly in solid form. In this process variant, too, a pyrolysis gas stream is obtained as product. The solid residue which is likewise obtained can, for example, be recycled to the pulping process.

In a specific embodiment, a black liquor material is used for the pyrolysis, which, under normal conditions (20° C., 1013 mbar), has a liquid content of not more than 70% by weight, particularly preferably of not more than 50% by weight, based on the total weight of the black liquor material.

The pyrolysis in step b) can, if desired, be carried out in the presence of at least one pyrolysis catalyst. Suitable pyrolysis catalysts are, for example, silica, alumina, aluminosilicates, aluminosilicates having sheet structures and zeolites such as mordenite, faujasite, zeolite X, zeolite Y and ZSM-5, zirconium oxide or titanium dioxide.

The temperature in the pyrolysis is preferably in the range from 200 to 1500° C., particularly preferably from 250 to 1000° C., in particular from 300 to 800° C.

The pressure in the pyrolysis is preferably in the range from 0.5 to 250 bar (absolute), preferably from 1.0 to 40 bar (absolute).

The residence time at the pyrolysis temperature can be from a few seconds to a number of days. In a specific embodiment, the residence time at the pyrolysis temperature is from 0.5 seconds to 5 minutes, especially from 2 seconds to 3 minutes. The residence time, especially in a fluidized-bed reactor, is given by the total volume of the reactor divided by the volume flow of the fluidizing gas under the pyrolysis conditions.

Suitable processes for the catalyzed or uncatalyzed pyrolysis of lignin are also described, for example, in WO 96/09350 (Midwest Research Institute, 1996) or U.S. Pat. No. 4,409,416 (Hydrocarbon Research Institute, 1983), which are hereby incorporated by reference.

In the pyrolysis zone, the biomass starting material, especially the lignin, is reacted to give a pyrolysis product which under the conditions of the pyrolysis is present at least partly in gaseous form (“pyrolysis gas”). Furthermore, a pyrolysis product which under the conditions of the pyrolysis is present partly in liquid and/or solid form can result from the pyrolysis.

The composition of the decomposition product obtained in step b) (=pyrolysis product) can vary as a function of the biomass used.

In all cases, the decomposition product obtained in the pyrolysis in step b) comprises substituted aromatics and/or polycyclic aromatics. The decomposition product can comprise, in addition to aromatics, further components selected from water vapor, inert gas (e.g. nitrogen), nonaromatic hydrocarbons, H₂, CO, CO₂, sulfur-comprising compounds such as H₂S, etc., and mixtures thereof. The nonaromatic hydrocarbons are preferably degradation products such as methane.

The separation and further treatment of the decomposition product obtained in the pyrolysis in step b) will be described in more detail in step c).

### 2nd Variant: Decomposition in the Liquid Phase

In a second variant of the process of the invention, the biomass starting material, especially the lignin-comprising starting material is subjected to decomposition in the presence of a liquid decomposition medium in step b). In this variant, the decomposition product is obtained at least partly in the liquid state.

The decomposition in the liquid state can be carried out by many methods which differ primarily in terms of the decomposition medium. The biomass starting material, especially the lignin-comprising starting material, is preferably subjected to decomposition in the presence of an aqueous-alkaline, aqueous-acidic or organic decomposition medium in step b).

Preference is given to using at least one cellulose-depleted fraction from a pulp process for the decomposition in the presence of a liquid decomposition medium in step b). In a specific embodiment, this cellulose-depleted fraction is a cellulose-depleted fraction from a pulp process which still comprises at least part of the liquid treatment medium from the digestion of the lignocellulose material for producing pulp.

The treatment medium used for the decomposition in step b) comprises at least one compound which is liquid under normal conditions (20° C. and 1013 mbar). This is preferably selected from water, acids, bases, organic solvents and mixtures thereof. Acids and bases which are liquid under normal conditions and liquid mixtures comprising acids or bases can be selected by a person skilled in the art from those mentioned below. The organic solvents are preferably selected from alcohols such as methanol, ethanol, n-propanol, isopropanol, n-butanol, tert-butanol or phenol, diols and polyols such as ethanediol and propanediol, amino alcohols such as ethanolamine, diethanolamine or triethanolamine, aromatic hydrocarbons such as benzene, toluene, ethylbenzene or xylenes, halogenated solvents such as dichloromethane, chloroform, carbon tetrachloride, dichloroethane or chlorobenzene, aliphatic solvents such as pentane, hexane, heptane, octane, ligroin, petroleum ether, cyclohexane or decalin, ethers such as tetrahydrofuran, diethyl ether, methyl tert-butyl ether or diethylene glycol monomethyl ether, ketones such as acetone or methyl ethyl ketone, esters such as ethyl acetate, formamide, dimethylformamide (DMF), dimethylacetamide, dimethyl sulfoxide (DMSO), acetonitrile and mixtures thereof.

The liquid compound is preferably selected from water, water-miscible organic solvents and mixtures thereof. The liquid compound is particularly preferably selected from water, alcohols and mixtures thereof. Thus, water, methanol, ethanol, a mixture of water with methanol and/or ethanol or a mixture of methanol and ethanol can be used as liquid compound.

The liquid decomposition medium used in step b) can comprise at least one base. Suitable bases are alkali metal and alkaline earth metal hydroxides, e.g. sodium hydroxide, potassium hydroxide, calcium hydroxide or magnesium hydroxide, alkali metal and alkaline earth metal hydrogencarbonates, e.g. sodium hydrogencarbonate, potassium hydrogencarbonate, calcium hydrogencarbonate or magnesium hydrogencarbonate, alkali metal and alkaline earth metal carbonates, e.g. sodium carbonate, potassium carbonate, calcium carbonate or magnesium carbonate, alkaline earth metal oxides such as calcium oxide or magnesium oxide, and mixtures thereof.

The liquid decomposition medium used in step b) can comprise at least one acid. Brönsted acids or Lewis are suitable in principle. Suitable Brönsted acids are inorganic acids, their acid salts and anhydrides. These include, for example, mineral acids such as hydrochloric acid, nitric acid, sulfuric acid, phosphoric acid or amidosulfonic acid, and also ammonium salts such as ammonium fluoride, ammonium chloride, ammonium bromide or ammonium sulfate. Further suitable acids are hydrogensulfates such as sodium hydrogensulfate, potassium hydrogensulfate, calcium hydrogensulfate or magnesium hydrogensulfate. Further suitable acids are hydrogensulfites such as sodium hydrogensulfite, potassium hydrogensulfite, calcium hydrogensulfite or magnesium hydrogensulfite. Further suitable acids are hydrogenphosphates and dihydrogenphosphates, e.g. sodium hydrogenphosphate, sodium dihydrogen-phosphate, potassium hydrogenphosphate or potassium dihydrogenphosphate. SO₂, SO₃ and CO₂ are also suitable.

Suitable Brönsted acids also include organic acids and anhydrides thereof, for example formic acid, acetic acid, methanesulfonic acid, trifluoroacetic acid or p-toluenesulfonic acid.

Suitable Lewis acids are all metal or semimetal halides in which the metal or semimetal has an electron pair vacancy. Examples are BF₃, BCl₃, BBr₃, AlF₃, AlCl₃, AlBr₃, ethylaluminum dichloride, diethylaluminum chloride, TiF₄, TiCl₄, TiBrₐ, VCl₅, FeF₃, FeCl₃, FeBr₃, ZnF₂, ZnCl₂, ZnBr₂, Cu(I)F, Cu(I)Cl, Cu(I)Br, Cu(II)F₂, Cu(II)Cl₂, Cu(II)Br₂, Sb(III)F₃, Sb(V)F₅, Sb(III)Cl₃, Sb(V)Cl₅, Nb(V)Cl₅, Sn(II)F₂, Sn(II)Cl₂, Sn(II)Br₂, Sn(IV)F₄, Sn(IV)Cl₄ and Sn(IV)Br₄.

The liquid decomposition medium used in step b) can comprise at least one salt different from the compounds mentioned above. The salts are preferably selected from salts of the abovementioned acids and bases and also their oxidation or reduction products. Suitable salts are, for example, ammonium, alkali metal or alkaline earth metal sulfates such as sodium sulfate, potassium sulfate, calcium sulfate or magnesium sulfate. Further suitable salts are ammonium, alkali metal or alkaline earth metal sulfites such as sodium sulfite, potassium sulfite, calcium sulfite or magnesium sulfite. Further suitable salts are ammonium, alkali metal or alkaline earth metal sulfides such as sodium sulfide, potassium sulfide, calcium sulfide or magnesium sulfide. Further suitable salts are alkali metal hydrogensulfides such as sodium hydrogensulfide or potassium hydrogensulfide.

The liquid decomposition medium used in step b) can comprise further compounds different from the abovementioned compounds. These are especially the customary process chemicals known to those skilled in the art in the various digestion processes for producing pulp from a lignocellulose-comprising starting material. Such processes and the process chemicals used therein are known to those skilled in the art.

In a first particularly preferred embodiment, an alkaline decomposition medium is used in step b). In particular, at least one cellulose-depleted fraction from a pulp process which comprises at least part of the alkaline digestion medium from the preceding pulp process is used for the decomposition in step b).

A cellulose-depleted fraction from the kraft process (sulfate process) is then preferably used for the decomposition in step b). The decomposition medium used in step b) then comprises NaOH and Na₂S in an aqueous medium. In a specific embodiment, the treatment medium used in step a) comprises NaOH, Na₂S, Na₂CO₃ and Na₂SO₄ in an aqueous medium.

In particular, a black liquor obtained in pulp production by the kraft process is used for the decomposition in step b). Here, it is possible to use either the weak black liquor obtained directly after the pulp fibers have been separated off or a concentrated quality obtained by evaporation. The decomposition in an alkaline aqueous phase as described by Clark and Green in Tappi, 51(1), 1968, 44 ff is particularly advantageous.

A cellulose-depleted fraction from the soda process (sodium carbonate process) can also be used for the decomposition in step b). The treatment medium used in step b) then comprises NaOH as main component in an aqueous medium which is essentially free of sulfur-comprising compounds.

A cellulose-depleted fraction from the alkali-oxygen digestion can also be used for the decomposition in step b).

A cellulose-depleted fraction from the alkali-peroxide digestion can also be used for the decomposition in step b).

A cellulose-depleted fraction from digestion in the presence of anthraquinone can also be used for the decomposition in step b).

A cellulose-depleted fraction from digestion of the lignocellulose material with organic solvents (also referred to as Organosolv process) can also be used for the decomposition in step b). Suitable organic solvents are those mentioned above. In particular, use is made of an organic solvent selected from C₁-C₄-alkanols, mixtures of C₁-C₄-alkanols and mixtures of at least one C₁-C₄-alkanol with water. The C₁-C₄-alkanols are preferably selected from methanol, ethanol, n-propanol, isopropanol and n-butanol. Preference is given to methanol, ethanol and mixtures thereof. Mixtures of at least one C₁-C₄-alkanol with water preferably comprise from 10 to 99% by weight, particularly preferably from 20 to 95% by weight, of at least one C₁-C₄-alkanol, based on the total weight of the mixture. The decomposition medium used in step b) can then additionally comprise an additive from the parent pulp process. Such additives include, for example, alkali metal hydroxides such as sodium hydroxide; ammonium hydrogensulfite and also alkali metal and alkaline earth metal hydrogensulfites such as sodium hydrogensulfite and magnesium hydrogensulfite. They also include mineral acids such as hydrochloric acid, nitric acid, sulfuric acid, phosphoric acid or amidosulfonic acid and their ammonium, alkali metal and alkaline earth metal salts. Organic acids such as oxalic acid, formic acid or acetic acid are also suitable as additives. Peracids such as persulfuric acid or peracetic acid are also suitable.

The cellulose-depleted fractions which contain at least part of the liquid treatment medium from one of the following commercially operated Organosolv processes are also suitable for use in step b) of the process of the invention:

Alcell process: ethanol/water mixture as treatment medium.

ASAM process: alkaline sulfite-anthraquinone-methanol treatment medium.

Organocell process: two-stage process using an organic medium in the first stage and an alkaline medium in the second stage, e.g. digestion using methanol and/or ethanol in the first stage and using methanol and/or ethanol, NaOH and optionally anthraquinone in the second stage.

Acetosolv process: acetic acid/hydrochloric acid mixture as treatment medium.

The decomposition in the presence of a liquid decomposition medium in step b) can be carried out in one or more stages. In the simplest case, the decomposition in step b) is carried out in one stage.

The decomposition in step b) is preferably carried out above ambient temperature. The temperature is preferably in the range from about 40 to 300° C., particularly preferably from 50 to 250° C. In a specific embodiment, the temperature is firstly increased stepwise or continuously during the course of the treatment until the desired final temperature has been reached.

The decomposition in step b) can be carried out under reduced pressure, at ambient pressure or above ambient pressure. The pressure in step a) is generally in the range from 0.01 bar to 300 bar, preferably from 0.1 bar to 100 bar.

The duration of the decomposition in step b) is generally from 0.5 minutes to 7 days, preferably from 5 minutes to 96 hours.

If a cellulose-depleted fraction from the pulp process is used for the decomposition in step b), then the decomposition is advantageously carried out in close proximity to the site of pulp production in order to keep the outlay for transport of the cellulose-depleted fraction, especially a black liquor, low. Transport is preferably effected via a pipe.

In all cases, the decomposition product obtained from the decomposition in the presence of a liquid decomposition medium in step b) comprises substituted aromatics and/or polycyclic aromatics.

The separation and further treatment of the decomposition product obtained in the presence of a liquid decomposition medium in step b) will be described in more detail in step c).

It is in principle possible to use the decomposition product obtained in step b) without further separation and/or treatment for the dealkylation in step d). If the digestion product obtained in step b) is obtained in the liquid state, it is preferably subjected to vaporization before being introduced into step d). A preferred embodiment of the vaporization is shown in FIG. 2 and described below.

In another embodiment of the process of the invention, the decomposition product obtained in step b) is subjected to a separation and/or treatment (step c) before being used in the dealkylation (step d).

### Separation/Treatment of the Decomposition Product (Step c)

The decomposed material obtained in step b) is preferably separated into at least one aromatics-enriched fraction C1) and at least one aromatics-depleted fraction C2) in step c).

The separation is preferably effected by distillation, extraction, absorption, membrane processes or a combination thereof. The separation is particularly preferably effected by distillation, extraction or a combination thereof.

If the decomposition in step b) is carried out in the liquid state, the separation in step c) is preferably carried out by means of distillation and/or extraction.

In a first specific embodiment of the process of the invention, the biomass starting material prepared in step a) is subjected in step b) to a decomposition in the liquid state and the separation into at least one aromatics-enriched fraction C1) and at least one aromatics-depleted fraction C2) in step c) comprises an extraction and/or a distillation.

Before the separation in step c), the pH of the discharge from a decomposition in the liquid state in step b) is preferably set to a desired value. In a specific embodiment, a decomposition product which has been obtained by decomposition in the presence of an alkaline digestion medium is used in step c). In particular, at least one cellulose-depleted fraction from a pulp process, in particular a black liquor from the kraft process, is used for the decomposition. The pH is then preferably set to a value of not more than 9, particularly preferably not more than 8, by addition of acid before the separation of the decomposition product. Suitable acids are, for example, mineral acids such as hydrochloric acid, sulfuric acid and phosphoric acid and also acid-forming compounds such as CO₂ and H₂S. Preference is given to using CO₂ from an offgas stream from the process of the invention or a pulp process coupled with the process of the invention. A suitable offgas is, for example, the offgas from a black liquor combustion (recovery boiler) or a lime kiln. Here, the offgas can be introduced into the decomposition product either directly or after removal of other components (e.g. by means of a scrubbing process such as a Benfield scrub). The carbonates and/or hydrogencarbonates obtained by CO₂ addition can in general easily be recycled, for example into a pulp process coupled to the decomposition process, for example into a black liquor previously removed to obtain lignin. The use of CO₂ for adjusting the pH is thus associated with lower costs than when other acids are used and also generally allows good integration into a pulp process. The hydroxyaromatics obtained in the decomposition in step b) are virtually entirely present as salts (phenoxides) at pH values above about 9. Effective isolation by distillation and/or extraction is aided by lowering the pH to a value of <9, preferably <8, beforehand.

The separation by distillation of the product obtained from the decomposition in the liquid state in step b) can be carried out by conventional methods known to those skilled in the art. Preference is given to a steam distillation, giving a distillate enriched in aromatics. In this procedure, the steam volatility of the aromatic fragments obtained in the decomposition in step b) is utilized to separate these off from the decomposition product. Preference is given to a multistage process in which the heat of condensation of the vapors from at least one stage is utilized for vaporization in another stage. The distillation product obtained is enriched in aromatics compared to the decomposition product used and is suitable, optionally after removal of the aqueous phase, as starting material for the dealkylation in step d).

The separation of the product obtained from the decomposition in the liquid state in step b) is preferably also effected by extraction. Here, at least part of the aromatics obtained in the decomposition in step b) is separated off, while the remaining residue (organic components low in aromatics, inorganic process chemicals, etc.) can be passed to a further work-up and/or thermal utilization, preferably within the process of the invention or an integrated pulp process coupled therewith.

For the extraction, it is possible to use a solvent (extractant) in which the aromatics obtained in the decomposition are soluble in a sufficient amount and which otherwise forms a miscibility gap with the decomposition product. The extractant can then be brought into intimate contact with the decomposition product obtained in step b), followed by a phase separation. The extraction can have one or more stages.

Suitable extractants are organic compounds such as aromatic or nonaromatic hydrocarbons, alcohols, aldehydes, ketones, amides, amines and mixtures thereof. These include, for example, benzene, toluene, ethylbenzene, xylenes; pentane, hexane, heptane, octane, ligroin, petroleum ether, cyclohexane, decalin, n-butanol, sec-butanol, tert-butanol, 1-pentanol, 1-hexanol, 1-heptanol, 1-octanol, methyl ethyl ketone and mixtures thereof.

The extraction can be carried out continuously or batchwise; see description in: K. Sattler, Thermische Trennverfahren, Wiley-VCH, third revised and expanded edition, July 2001. A plurality of batch separation operations can be carried out one after the other in the manner of a cascade, with the residue separated off from the extractant phase in each case being brought into contact with a fresh portion of extractant and/or the extractant being conveyed in countercurrent. For batch operation, the decomposition product and the extractant are brought into contact with mechanical agitation, e.g. by means of stirring, in a suitable vessel, the mixture is allowed to stand for the phases to separate and one of the phases is removed, advantageously by taking off the heavier phase at the bottom of the vessel. To carry out the extraction continuously, the extractant and the decomposition product are continuously conveyed through a suitable apparatus in a manner analogous to the batchwise variant.

The extraction is carried out, for example, in at least one mixer-settler combination or at least one extraction column. Suitable mixers include both dynamic mixers and static mixers.

In a preferred embodiment, the separation into at least one aromatics-enriched fraction C1) and at least one aromatics-depleted fraction C2) in step c) comprises the following substeps:
c1) extraction of the decomposition product obtained in step b) to give an aromatics-enriched extract and an aromatics-depleted residue,
c2) optionally separation of the extract into an extractant-enriched (and aromatics-depleted) fraction and an aromatics-enriched (and extractant-depleted) fraction,
c3) introduction of the aromatics-enriched extract obtained in step c1) or the aromatics-enriched fraction obtained in step c2) into step d).

Before the extraction, the pH of the decomposition product obtained in step b) can be adjusted by addition of at least one acid or at least one base. Furthermore, in a multistage extraction, both the pH of the decomposition product used in the first stage and the pH of the residue separated off from the extractant phase in the respective stage can be adjusted by addition of at least one acid or acid-forming compound or at least one base. Suitable acids are, for example, mineral acids such as hydrochloric acid, sulfuric acid and phosphoric acid or acid-forming compounds such as CO₂ and H₂S. Suitable bases are, for example, alkali metal bases such as sodium hydroxide or potassium hydroxide, alkali metal carbonates such as sodium carbonate or potassium carbonate, alkali metal hydrogencarbonates such as sodium hydrogencarbonate or potassium hydrogencarbonate and alkaline earth metal bases such as calcium hydroxide, calcium oxide, magnesium hydroxide or magnesium carbonate, and also ammonia or amines.

The separation of the extract in step c2) into an extractant-enriched fraction and an aromatics-enriched fraction C1) is preferably carried out by distillation.

The separation by distillation of the extract in step c2) can be carried out by conventional methods known to those skilled in the art. Suitable processes are described in: K. Sattler, Thermische Trennverfahren, Wiley-VCH, third revised and expanded edition, July 2001. Suitable apparatuses for the separation by distillation comprise distillation columns such as tray columns which may be provided with bubble caps, sieve plates, sieve trays, packings, internals, valves, side offtakes etc. Dividing wall columns, which may be provided with side offtakes, recycling loops etc., are especially suitable. A combination of two or more than two distillation columns can be used for the distillation. Also suitable are evaporators such as thin film evaporators, falling film evaporators, Sambay evaporators etc. and combinations thereof.

If the digestion in step b) comprises a pyrolysis, the separation in step c) is preferably effected by means of absorption.

In a second embodiment of the process of the invention, the biomass starting material provided in step a) is subjected to a pyrolysis for the decomposition in step b) and the separation in step c) into at least one aromatics-enriched fraction C1) and at least one aromatics-depleted fraction C2) comprises an absorption.

The discharge taken off from the pyrolysis zone can comprise not only the pyrolysis gases but also proportions of solid and/or liquid components. These can be, for example, relatively nonvolatile components formed in the pyrolysis (e.g. carbonaceous material). If at least one solid inert material is used for the pyrolysis in step b), the discharge from the pyrolysis zone can also comprise proportions of the inert material. These solid and/or liquid components can, if desired, be separated off from the pyrolysis gas by means of a suitable apparatus, e.g. a cyclone, in step c). Solid inert materials which have been separated off are preferably recycled to the pyrolysis zone. Components other than inert materials which have been separated off are passed to another use, for example, combustion to obtain heat, which is preferably used further in the process of the invention or an integrated process. The offgas obtained in the process, comprising predominantly CO₂ and water and optionally O₂, can likewise be passed to another use. It is also possible to bring a discharge from the pyrolysis zone which comprises at least one inert material and components which are relatively nonvolatile under the pyrolysis conditions into contact with an oxygen-comprising gas, preferably air, in a burning-off zone which is physically separate from the pyrolysis zone, leading to burning-off of relatively nonvolatile components (“carbonaceous deposits”) formed in the pyrolysis. The inert material is then separated off from the offgas formed by means of a suitable separation apparatus and recycled by means of a suitable transport device to the pyrolysis zone.

In a useful embodiment, the discharge from the pyrolysis can be fed directly, i.e. without removal of condensable components, into the subsequent dealkylation zone. In this embodiment, however, components of the discharge from the pyrolysis zone which are relatively nonvolatile under the conditions of the pyrolysis in step b) and are not present in gaseous form but in solid or liquid form in the discharge from the pyrolysis zone can be separated off before introduction into the dealkylation zone. In a particular embodiment, on the other hand, condensable pyrolysis products (i.e. products which are present as liquids or solids under normal conditions) are separated off from the discharge from the pyrolysis (after solids/liquid have been separated off). This can be effected by means of suitable separation processes known to those skilled in the art, e.g. condensation, absorption, adsorption, membrane separation processes, etc.

A particularly preferred variant is an absorption d1. In this case the discharge from the pyrolysis zone is brought into contact with a stream D1) comprising a suitable solvent. The contacting is preferably effected after a cooling step in which a condensation of high-boiling components can also take place. The contacting is effected in a suitable apparatus (e.g. a column). A liquid stream D2) comprising the absorption medium and aromatic pyrolysis products and a gaseous stream D3) which is depleted in aromatic pyrolysis products compared to the discharge from the pyrolysis flow out of the contact apparatus. Stream D2) is separated, preferably by distillation, into a fraction D4) which is enriched in aromatic pyrolysis products compared to D2) and a fraction D5) which is depleted in aromatic pyrolysis products compared to D2). D4) is, if necessary after further work-up, fed as stream C1) into the subsequent dealkylation step and D5) is, after further cooling, recycled to the absorption, i.e. D5) is the main constituent of D1). A further constituent is a portion of solvent which is added to make up solvent losses.

Solvents suitable as absorption medium are organic compounds such as aromatic or nonaromatic hydrocarbons, alcohols, aldehydes, ketones, amides, amines and mixtures thereof. They include, for example, benzene, toluene, ethylbenzene, xylenes; pentane, hexane, heptane, octane, ligroin, petroleum ether, cyclohexane, decalin, methanol, ethanol, n-propanol, isopropanol, n-butanol, sec-butanol, tert-butanol, 1-pentanol, 1-hexanol, 1-heptanol, 1-octanol, acetaldehyde, acetone, methyl ethyl ketone, N-methylpyrrolidone, dimethylformamide, dimethylacetamide and mixtures thereof.

The solvent preferably has a boiling point which is below that of the phenol under identical conditions. The solvent particularly preferably has a boiling point which is at least 10 K below that of the phenol under identical conditions. The solvent preferably additionally has a high solubility in water. Such solvents include, for example, methanol, ethanol, n-propanol, isopropanol, n-butanol, sec-butanol and tert-butanol.

Many of the solvents used as absorption medium have a vapor pressure under the conditions of the absorption which leads to loading of the gas stream D3) leaving the absorption with the absorption medium. This applies especially to the preferably used solvents having a boiling point below the boiling point of phenol. The gas stream D3) exiting from the absorption is then preferably subjected to an at least partial removal of the solvent comprised. The separation of the solvent from the gas stream D3) is preferably carried out in the form of a water scrub. Here, good solubility in water of the solvent used for the absorption is particularly advantageous. The scrubbing water stream loaded with solvent and optionally aromatics can, for example, be worked up by distillation. The absorption medium separated off here is (optionally together with the aromatics) returned to the absorption step d1).

The decomposition product obtained in step b) can be subjected in step c) not only to the above-described separation but also to at least one further treatment step. Additional treatment steps can be carried out before, during or after the separation.

The decomposition product obtained in step b) or the fraction C1) isolated therefrom in step c) preferably comprises predominantly components having a molecular weight of not more than 500 g/mol, particularly preferably not more than 400 g/mol, in particular not more than 300 g/mol.

In a specific embodiment of the process of the invention, at least part of the aromatics-depleted fraction C2) isolated in step c) is used for producing synthesis gas.

### Dealkylation (Step d)

In the dealkylation, the aromatic degradation products formed in the pyrolysis in step b) and optionally isolated as fraction C1) in step c) are at least partly transformed by action of hydrogen and/or water vapor so that substituents are replaced by hydrogen and/or compounds comprising a plurality of aromatic rings are cleaved to form compounds having a smaller number of rings. As indicated above, the term “dealkylation” also refers to reactions in which no alkyl substituent is replaced by hydrogen, e.g. dehydroxylation, dealkoxylation, aromatics cleavage, etc. The substituents replaced by hydrogen are preferably selected from alkyl groups, hydroxy groups and alkoxy groups.

Dealkylation processes suitable for use in step d) comprise hydrodealkylation, steam dealkylation or mixed forms derived therefrom. In the case of a pure hydrodealkylation in the context of the invention, molecular hydrogen (in pure form or as a mixture with other components, e.g. CO) but no water is fed into the dealkylation zone in addition to the pyrolysis gas stream. In the case of a pure steam dealkylation in the context of the invention, water (in pure form or as a mixture with other components) but no molecular hydrogen is fed into the dealkylation zone in addition to the pyrolysis gas stream. The dealkylation process in step d) can also be configured as a mixed form of hydrodealkylation and steam dealkylation. Both water and molecular hydrogen are then fed into the dealkylation zone in addition to the pyrolysis gas stream. Suitable and preferred process parameters, partly for hydrodealkylation and partly for steam dealkylation, are indicated below. Using this information, a person skilled in the art will be able to determine suitable and preferred process parameters for a mixed form of hydrodealkylation and steam dealkylation. The reaction gas comprising H₂ and H₂O used for the dealkylation then preferably has a mixing ratio of H₂ to H₂O in the range from about 0.1:99.9 to 99.9:0.1. An especially suitable mixing ratio of H₂ to H₂O is in the range from about 40:60 to 60:40.

The hydrogen required for the reaction is, in the case of the steam dealkylation, formed in-situ by reaction of water with (mainly organic) components which are either comprised in the starting mixture for the steam dealkylation or are formed during the steam dealkylation. An example which may be mentioned here is the formation of hydrogen from methane and water according to the equation CH₄+H₂O→CO+3H₂.

The temperature in the dealkylation zone is preferably in the range from 400 to 900° C., particularly preferably from 500 to 800° C.

The absolute pressure in the dealkylation zone is preferably in the range from 1 to 100 bar, particularly preferably from 1 to 20 bar.

In a first preferred embodiment, the pyrolysis gas stream is subjected to a hydrodealkylation in step c). For this purpose, the reaction in step c) is carried out in the presence of hydrogen.

The temperature in the dealkylation zone for the hydrodealkylation is preferably in the range from 500 to 900° C., particularly preferably from 600 to 800° C.

The absolute pressure in the dealkylation zone for the hydrodealkylation is preferably in the range from 1 to 100 bar, particularly preferably from 1 to 20 bar, in particular from 1 to 10 bar.

The ratio of the amount of H₂ used to H₂ (stoichiometric) in the hydrodealkylation is preferably in the range from 0.02 to 50, particularly preferably from 0.2 to 10. Here, H₂ (stoichiometric) is the minimum amount of H₂ which is theoretically required for complete conversion of the aromatics fed into the dealkylation zone into benzene, with the assumption that 1 mol of H₂ reacts per ring substituent.

The residence time in the dealkylation zone for the hydrodealkylation is preferably in the range from 0.1 to 500 s, particularly preferably from 0.5 to 200 s.

In a second preferred embodiment, the pyrolysis gas stream is subjected to a steam dealkylation in step c). For this purpose, the reaction in step c) is carried out in the presence of water vapor.

The temperature in the dealkylation zone for the steam dealkylation is preferably in the range from 400 to 800° C., particularly preferably from 475 to 600° C., in particular from 525 to 600° C.

The absolute pressure in the dealkylation zone for the steam dealkylation is preferably in the range from 1 to 100 bar, particularly preferably from 1 to 20 bar, in particular from 1 to 10 bar.

The ratio of the amount of H₂O used to C* in the steam dealkylation is preferably in the range from 0.1 to 20 mol/mol, particularly preferably from 0.5 to 2 mol/mol. C* is the molar amount of carbon determined by carbon-based balancing of the pyrolysis or by determination of the amounts of product discharged from the steam dealkylation by means of methods known to those skilled in the art.

The molar ratio of H₂ to CH₄ in the dealkylation zone of the steam dealkylation is preferably <50:1, particularly preferably <25:1.

In a steam dealkylation in the absence of a dealkylation catalyst, the molar ratio of OR (where R═H, alkyl) to Cₜₒₜₐₗ in the dealkylation zone is preferably >0.05:1, particularly preferably from 0.1:1 to 0.2:1.

In a steam dealkylation in the absence of a dealkylation catalyst, the ratio of OR (where R═H, alkyl) to C_{eliminable} in the dealkylation zone is preferably >0.5:1, particularly preferably from 1:1 to 10:1, in particular from 1:1 to 2:1.

The WHSV in the steam dealkylation is preferably in the range from 0.05 to 10 kg/l*h, particularly preferably from 0.1 to 2 kg/l*h.

The steam dealkylation can be carried out in the presence or absence of a catalyst. In a specific embodiment, the steam dealkylation is carried out in the absence of a catalyst. A catalyzed process for steam dealkylation is described in WO 2008/148807 A1. This document and the references cited therein is, in respect of suitable catalysts, hereby fully incorporated by reference. Further information on catalyst types and process steps for steam dealkylation may be found in WO 2007/051852 A1, WO 2007/051851 A1, WO 2007/051855 A2, WO 2007/051856 A1, WO 2008/135581 A1 and WO 2008/135582 A1 (EP 2008055585), without being restricted thereby. U.S. Pat. No. 3,775,504 states that a steam dealkylation actually comprises a combination of steam dealkylation and hydrodealkylation, since it is inherent in the system that at least part of the hydrogen produced is immediately reacted again.

At least one low molecular weight aromatic material of value is formed as target product of the process of the invention in the dealkylation step d). The low molecular weight aromatic materials of value are preferably selected from benzene and phenolic compounds such as phenol and/or dihydroxybenzenes.

They have, in particular, smaller proportions of the following components than the pyrolysis discharge before introduction into the dealkylation step c): monoalkylated, dialkylated and polyalkylated phenols; alkoxyphenols such as methoxyphenols; polyalkylated benzenes; compounds comprising two or more aromatic rings. These components will hereinafter be referred to as “aromatics which are not dealkylated or have a low degree of dealkylation”.

Separation of the Discharge from the Dealkylation Zone (Step e)

A discharge is taken from the dealkylation zone and subjected to a separation. Here, at least one organic liquid or liquefiable material of value is obtained as first product of value and at least one stream enriched in components which are more volatile than the organic material of value is obtained as second product of value. Preference is given to obtaining an aromatics composition having a high content of single-ring aromatics which are unalkylated or have a low degree of alkylation as first product of value.

The discharge from the dealkylation zone is preferably subjected to a separation to give the following three streams:
E1) a stream enriched in single-ring aromatics which are unalkylated or have a low degree of alkylation,
E2) a stream enriched in aromatics which are not dealkylated or have a low degree of dealkylation,
E3) a stream enriched in by-products which are more volatile than E1) and E2).

The discharge from the dealkylation zone can optionally be subjected to a separation to give further streams, e.g. a water-comprising stream.

Stream E1) is the first product of value produced in the process of the invention. E1) is preferably an aromatics composition having a high content of single-ring aromatics which are unalkylated or have a low degree of alkylation. In addition, stream E1) can be subjected to a further work-up to give the aromatics composition produced according to the invention.

Stream E1) preferably comprises at least 70% by weight, particularly preferably at least 80% by weight, in particular at least 90% by weight, based on the total amount of E1), of single-ring aromatics.

Stream E1) preferably comprises not more than 30% by weight, particularly preferably not more than 20% by weight, in particular not more than 10% by weight, based on the total amount of E1), of aromatics which are not dealkylated or have a low degree of dealkylation.

Stream E2) preferably comprises at least 70% by weight, particularly preferably at least 80% by weight, in particular at least 90% by weight, based on the total amount of E2), of aromatics which are not dealkylated or have a low degree of dealkylation.

Stream E3) comprises components which are, for example, selected from nonaromatic hydrocarbons, especially methane, hydrogen, carbon monoxide, carbon dioxide and mixtures thereof. Depending on the lignin-comprising starting material provided in step a), the stream E3) can comprise further components. When a lignin-comprising starting material from the kraft process is used, such further components include sulfur-comprising by-products, especially H₂S.

Preference is given to taking a gaseous discharge from the dealkylation zone and subjecting it to a separation in step e).

As process for the separation, it is possible to use the generally known thermal separation processes.

The separation of the discharge from the dealkylation zone in step e) preferably comprises an absorption. In the absorption, the gaseous discharge from the dealkylation zone is brought into contact with a solvent (absorption medium), with part of the components comprised in the gas stream being absorbed and thus separated off.

The absorption is carried out in a suitable apparatus, e.g. a countercurrent column, bubble column, etc. The absorption is preferably carried out in a countercurrent column.

The absorption can have one or more stages.

The absorption is preferably carried out using a solvent (not loaded: absorbent, loaded: absorbate) in which the aromatics obtained in the dealkylation are soluble in a sufficient amount and the relatively volatile by-products different therefrom are essentially insoluble. The aromatics which are not dealkylated or have a low degree of dealkylation are at least partly absorbed together with the single-ring aromatics which are unalkylated or have a low degree of alkylation (=target product).

The absorption thus gives an absorbate loaded with aromatics. The aromatic components comprised in the absorbate correspond in terms of their composition to the sum of the aromatics in streams E1) and E2) plus optionally aromatics comprised in the absorption medium. The components comprised in the remaining gas stream correspond in terms of their composition to stream E3). If desired, the gas stream can be subjected to an additional purification step to remove aromatics. These can then once again be combined with the aromatics comprised in the solvent separated off for joint work-up. However, such an isolation of aromatics from the gas stream separated off is generally not necessary.

In a preferred embodiment, the separation of the discharge from the dealkylation zone in step e) comprises the following substeps:
e1) contacting of the discharge from the dealkylation zone with an absorption medium to give an absorbate enriched in aromatic primary products from the dealkylation and a gas stream E3) depleted in aromatic primary products from the dealkylation,
e2) separation of the absorbate into a stream E1) enriched in single-ring aromatics which are unalkylated or have a low degree of alkylation, a stream E2) which is enriched in aromatics which are not dealkylated or have a low degree of dealkylation and optionally a stream comprising the absorption medium,
e3) if present, recycling of the stream comprising the absorption medium to step e1),
e4) optionally recycling of at least part of the stream E2) to the dealkylation zone of step d).

The absorption medium preferably has a boiling point which is above the boiling point of the highest-boiling components of stream E1.

In a first suitable embodiment, use is made of an absorption medium which is different from the components of streams E1) and E2). Suitable absorption media for this embodiment are nonaromatic hydrocarbons, nonaromatic alcohols, nonaromatic aldehydes, ketones, amides, amines and mixtures thereof. The absorption medium for this embodiment is preferably selected from pentane, hexane, heptane, octane, ligroin, petroleum ether, cyclohexane, decalin, methanol, ethanol, n-propanol, isopropanol, n-butanol, sec-butanol, tert-butanol, 1-pentanol, 1-hexanol, 1-heptanol, 1-octanol, acetaldehyde, acetone, methyl ethyl ketone, N-methylpyrrolidone, dimethylformamide, dimethylacetamide and mixtures thereof.

Further suitable absorption media are aromatic hydrocarbons different from the components of streams E1) and E2). These aromatic hydrocarbons are preferably selected from optionally substituted acetophenones, optionally substituted benzophenones, optionally substituted biphenyls, optionally substituted terphenyls, optionally substituted diphenyl ethers and mixtures thereof. If a component which is also comprised as by-product in stream E1) or E2) is used as absorption medium, then it can be ensured by means of instrumentation measures known to those skilled in the art that this component is continually discharged from the process in the amount in which it is obtained.

When an absorption medium which is different from the components of streams E1) and E2) is used, the separation of the discharge from the dealkylation zone in step e) preferably comprises the following substeps:
e1) contacting of the discharge from the dealkylation zone with an absorption medium to give an absorbate enriched in aromatic primary products from the dealkylation and a gas stream E3) depleted in aromatic primary products from the dealkylation (or enriched in by-products which are more volatile than E1 and E2),
e2) separation of the absorbate into a stream E1) enriched in single-ring aromatics which are unalkylated or have a low degree of alkylation, a stream E2) which is enriched in aromatics which are not dealkylated or have a low degree of dealkylation and a stream comprising the absorption medium,
e3) recycling of the stream comprising the absorption medium to step e1),
e4) optionally recycling of at least part of the stream E2) to the dealkylation zone of step d).

In a particularly preferred variant, an aromatics composition which can be obtained by the process of the invention is used as absorption medium. This is especially a mixture of aromatics which have not been reacted or have been incompletely reacted in the dealkylation. In a particularly preferred variant, the absorption medium used is an aromatics composition whose composition corresponds partially or completely to stream E2 or a mixture of E1 and E2. Optionally the stream E2 or the mixture of E1 and E2 can be subjected to at least one work-up step before being used as absorption medium.

When an absorption medium whose composition corresponds largely or totally to stream E2 or a mixture of E1 and E2, the separation of the discharge from the dealkylation zone in step e) preferably comprises the following substeps:
e1) contacting of the discharge from the dealkylation zone with an absorption medium to give an absorbate enriched in aromatic primary products from the dealkylation and a gas stream E3) depleted in aromatic primary products from the dealkylation,
e2) separation of the absorbate into a stream E1) enriched in single-ring aromatics which are unalkylated or have a low degree of alkylation and a stream E2) which is enriched in aromatics which are not dealkylated or have a low degree of dealkylation,
e4) optionally recycling of at least part of the stream E2) to the dealkylation zone of step d).

In this variant, the solvent can be obtained by partial condensation of the stream from the dealkylation or a gas stream from a preliminary removal of high boilers downstream of the dealkylation. Here, it can be advantageous to insert a further partial condensation in which, in particular, water is condensed out between the abovementioned partial condensation and the absorption. In this variant too, at least partial absorption of the unreacted or incompletely reacted aromatics takes place together with the absorption of the product of value. This means that in this variant, too, the aromatic components comprised in the absorbate correspond in their composition to the sum of the aromatics of streams E1) and E2).

In step e2), the aromatics-enriched absorbate is preferably separated by distillation. The solvent recovered here is, optionally after removal of absorbed water, recycled to the absorption (step e1). The aromatics are processed further as described above and below.

In step e2), the aromatics-enriched absorbate is separated by distillation in at least one column (“regeneration column”). The distillation conditions are preferably selected so that essentially aromatics which are unalkylated or have a low degree of alkylation and, if present, water are obtained as overhead product and essentially the aromatics which are not dealkylated or have a low degree of dealkylation are obtained as bottom product.

It goes without saying that the temperature at the bottom in the separation by distillation in step e2) is selected at such a low value that undesirable secondary reactions of the bottom product are essentially avoided. This can, in particular, be achieved by setting a suitable column pressure and/or the low boiler content in the bottoms (the low boiler content can be reduced further by downstream stripping).

The overhead product obtained in the distillation in step e2) comprises the target product of the process of the invention. It can either be taken off directly as stream E1) or be subjected to a further work-up. Water comprised in the overhead product can be separated off by known methods. The overhead product after condensation of the vapors from the distillation can for this purpose be fed to a phase separator to separate off the water. The resulting water is discharged as a further stream from the process. The organic phase from the phase separator can either be taken off at least partly as stream E1) or be subjected to a further work-up. The organic phase from the phase separator can partly be recycled as runback to the column and/or be subjected to a further work-up by distillation. This preferably serves to remove water still present and/or undesirable organic components.

The bottom product obtained in the distillation in step e2) comprises the aromatics which have not been reacted or not been sufficiently reacted in the dealkylation, i.e. it is enriched in aromatics which are not dealkylated or have a low degree of dealkylation. It can either be taken off directly as stream E2) or be subjected to a further work-up. The bottom product obtained in the distillation in step e2) is preferably divided into at least two substreams. In step e), a first substream is preferably recycled as absorption medium to the absorptive separation of the discharge from the dealkylation zone. For this purpose, this substream is, if necessary, cooled to a suitable temperature. A second substream is taken off as stream E2). The stream E2 can be subjected to a removal of constituents which do not belong to stream D2 before recycling to the dealkylation zone of step c). This is advantageous when, for example, an absorption solvent which is not obtained as intermediate in the process of the invention is used. In addition, it is advantageous also to take off a purge stream from stream E2) at this point and pass this purge stream to, for example, an incineration apparatus in order to reduce the accumulation of components which do not react or react slowly under the conditions of the dealkylation.

The stream E2) is preferably subjected to vaporization before being fed into the dealkylation. A preferred variant is shown in FIG. 2 and explained in the associated description of the figures.

According to the invention, the stream E3) obtained in step e), which is depleted in aromatics and enriched in volatile by-products, is at least partly used for producing synthesis gas. When, according to the above-described embodiment of the process of the invention, the separation of the discharge from the dealkylation zone in step e) comprises an absorption, the gas stream leaving the absorption apparatus (stream E3) is, optionally after a purification step to remove absorption medium and/or aromatics, at least partly used for producing synthesis gas.

The stream E3) obtained in step e) can, in addition to synthesis gas production, be partly fed to various other uses. These include combustion. If the process of the invention is in the physical proximity of a pulp process, it can be advantageous to feed stream E3) into an apparatus of the pulp process. Particular preference is given to feeding the stream E3) into the waste liquor combustion (recovery boiler). This embodiment has the advantage that no additional apparatuses for steam or power generation or flue gas desulfurization for combustion of the stream E3) are required. In another variant, the combustion of the stream E3) is preceded by a desulfurization, e.g. in the form of a gas scrub to remove hydrogen sulfide, followed by conversion of the H₂S formed into elemental sulfur. The formation of sulfur can be carried out by known processes, e.g. the Claus process. Instead, the combustion can also be followed by a desulfurization unit.

At least one further stream comprising, for example, water vapor and/or oxygen can optionally be used in addition to stream E3) for the production of synthesis gas.

In a specific embodiment of the process of the invention, at least part of the aromatics-depleted fraction C2) isolated in step c) is used for producing synthesis gas. It is also possible to use an offgas stream from the decomposition in step b) and/or the dealkylation in step d) in the production of synthesis gas. This offgas stream can be, for example, a burning-off gas from the combustion of relatively nonvolatile components. The introduction of such an offgas stream enables the H₂/CO ratio of the synthesis gas to be reduced.

The production of synthesis gas preferably comprises the following stages:
a reforming stage,
a converting stage (into which additional water is introduced if necessary) in which the water gas shift reaction (CO+H₂O [symbol] H₂+CO₂) occurs,
optionally a stage for the partial removal of acidic gases such as CO₂.

The way in which the production of synthesis gas is carried out corresponds to the prior art as described, for example, in Ullmann's Encyclopedia of Industrial Chemistry, article “Gas Production”, DOI: 10.1002/14356007.a12_{—}169.pub2.

In a preferred variant, all or part of the synthesis gas produced in the process of the invention is (if necessary after further purification steps known per se to remove water, sulfur-comprising components, CO₂, etc.) used in at least one process which consumes hydrogen, CO or mixtures of the two. Such a process can be, for example, a hydrogenation, hydroformylation, carbonylation, methanol synthesis, synthesis of hydrocarbons by the Fischer-Tropsch process, etc.

In a preferred embodiment of the process of the invention, a synthesis gas-comprising stream produced in the process or a hydrogen-enriched stream produced from the synthesis gas is fed into the decomposition in step b) and/or into the dealkylation in step d). Enrichment of the synthesis gas in hydrogen can, as described above, be effected by means of the water gas shift reaction.

Preference is given to feeding a synthesis gas-comprising stream produced in the process or a hydrogen-enriched stream produced from the synthesis gas into the dealkylation in step d). The particular advantage of this variant is that the proportion of phenol(s) in the products of the dealkylation is higher than in the pure steam dealkylation, i.e. without introduction of hydrogen. The higher phenol formation represents an economic advantage since phenol is a more valuable material than oxygen-free aromatics such as benzene and also retains more of the mass of the starting material lignin. In addition, hydrogen which is not produced in the process of the invention is more expensive and in many cases unavailable or obtainable only with a high degree of difficulty, especially when the dealkylation is to be carried out away from an integrated chemical site.

### DESCRIPTION OF FIGURES

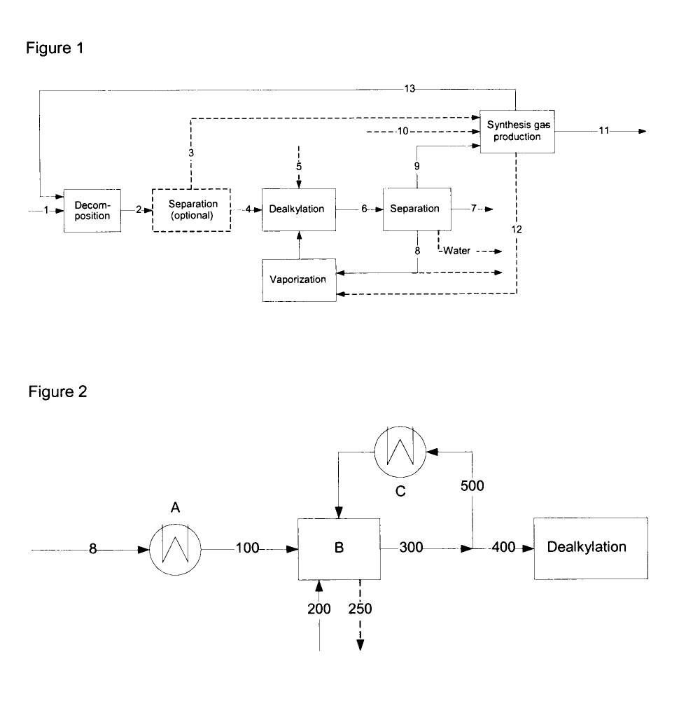

A preferred embodiment of the process of the invention is shown in FIG. 1.

A biomass starting material (1), in particular a lignin-comprising starting material (1), is subjected to a decomposition.

The decomposition product (2) is optionally subjected to a separation and/or treatment in which an aromatics-enriched stream (4) and an aromatics-depleted stream (3) are obtained. The aromatics-depleted stream (3) is optionally fed to reforming/converting for synthesis gas production.

The decomposition product (2) or the aromatics-enriched stream (4) obtained therefrom is fed together with a hydrogenation gas stream (5) to a dealkylation unit. The discharge from the dealkylation zone (6) is subjected to a separation into the following three streams:
product of value (stream 7), a material or mixture which has been formed in the above-described dealkylation;
product which has not been dealkylated or has been incompletely dealkylated (stream 8). This comprises materials which have not been dealkylated or have been dealkylated to a lesser extent than the product of value;
stream 9 comprising relatively volatile by-products. These are selected from methane and other hydrocarbons, H₂O, CO, CO₂ and sulfur-comprising by-products (in the case of lignin from the kraft process especially H₂S).

A water stream is optionally separated off from the discharge from the dealkylation zone (6) and discharged.

Stream (7) is, optionally after further work-up, taken off as product stream.

The stream (8) enriched in aromatics which are not dealkylated or have a low degree of dealkylation is returned to the dealkylation via a vaporization. A preferred embodiment of the vaporization is depicted in FIG. 2 and described in more detail below.

The stream (9) comprising volatile by-products from the separation is at least partly fed to reforming/converting to produce synthesis gas. Here, optionally with the introduction of a stream (10) comprising water or oxygen, the organic components comprised in stream (9) are converted into a synthesis gas (11) comprising CO and H₂.

A substream of stream (9) can also be fed to various other uses, e.g. a suitable combustion; when in the spatial vicinity of a pulp process, it is advantageous to feed stream (9) into an apparatus thereof, particularly preferably into the waste liquor combustion (recovery boiler). This embodiment has the advantage that no additional apparatuses are required for steam or power generation or flue gas desulfurization. In another variant, the combustion is preceded by a desulfurization, e.g. in the form of a hydrogen sulfide-removing gas scrub, followed by conversion of the H₂S into elemental sulfur (e.g. Claus process).

If the dealkylation is a hydrodealkylation, a hydrogen-comprising stream (12) obtained from synthesis gas production can be fed to the dealkylation.

If hydrogen is used for the decomposition, a hydrogen-comprising stream (13) obtained from synthesis gas production can be fed to the decomposition.

FIG. 2 shows the vaporization of an aromatics-comprising stream as is obtained, for example, as stream E2) (denoted by (8) in FIG. 1) in the separation by absorption and distillation of the discharge from the dealkylation zone. Stream (8) is preferably subjected to vaporization as shown in FIG. 2) before recycling to the dealkylation.

The aromatics stream (8) is preheated in apparatus A to a temperature at which no appreciable decomposition yet occurs in the liquid phase and this preheated stream (stream 100) is fed together with a gaseous stream (stream 200) whose amount, temperature and composition are selected so that the stream 100 partially or fully vaporizes into an apparatus B. The stream 200 comprises reactants for the dealkylation, i.e. in the case of steam dealkylation water vapor and in the case of hydrodealkylation a hydrogen-comprising gas (stream 5 in FIG. 1)). The amounts of streams 100 and 200 are set so that a composition favorable for the type of dealkylation selected is obtained in the stream 300 leaving the apparatus B.

Apparatus B is configured as a liquid-gas contact apparatus according to the prior art, for example, as a vessel having a jet nozzle or a column, with stream 100 being introduced at the top and liquid and gas being conveyed in concurrent or countercurrent; a relatively nonvolatile residue (stream 250) can be taken off in the lower part if necessary. As an alternative, apparatus B can also be configured as a fluidized bed. Additional energy can be introduced efficiently into the stream 100 via the externally heated fluidized material.

In a preferred variant, stream 300 is divided into streams 400 and 500, with stream 400 being recycled to the dealkylation and stream 500 being recycled via a heat exchanger C to apparatus B. This variant allows the temperatures of the streams 100, 200 and 500 (downstream of the heat exchanger) to be limited to limiting values determined by the availability of the heat sources, the thermal stability of the materials in the streams and the stability of the materials of construction. The pressure drop which naturally occurs along the streams 300, 400 and 500 is compensated by means of a suitable apparatus for compression. Here, it is possible to use generally known compressors or blowers, but it is also possible to configure the apparatus B completely or partially as a liquid jet blower, with stream 100 being used as driving medium. In this case, it is possible, if the amount of stream 100 is not sufficient for the compressing power required, to circulate liquid via apparatus B.

1. A process for producing synthesis gas and at least one organic liquid or liquefiable material of value, wherein
a) a biomass starting material is provided,
b) the biomass starting material is subjected to a decomposition,
c) the decomposed material obtained in step b) is optionally separated into at least one aromatics-enriched fraction C1) and at least one aromatics-depleted fraction C2),
d) the decomposition product from step b) or the aromatics-enriched fraction C1) from step c) is fed into a dealkylation zone and reacted in the presence of hydrogen and/or water vapor,
e) a discharge is taken from the dealkylation zone and subjected to a separation to give at least one organic liquid or liquefiable material of value and at least one stream enriched in components which are more volatile than the organic material of value,
f) the stream enriched in components which are more volatile than the organic material of value which is obtained in step e) is at least partly used for producing synthesis gas.

2. The process according to claim 1, wherein, in step a), a lignin-comprising material is provided as biomass starting material.

3. The process according to either of the preceding claims, wherein, in step a), a lignocellulose material or a digestion product from a lignocellulose material is provided as biomass starting material.

4. The process according to any of the preceding claims, wherein, in step a), a lignin-comprising stream from the digestion of a lignocellulose material for producing cellulose (pulp), preferably a black liquor, in particular a black liquor from the kraft digestion (sulfate digestion) is provided as biomass starting material.

5. The process according to any of claims 1 to 4, wherein, in step b), the biomass starting material provided is subjected to a pyrolysis to effect decomposition.

6. The process according to claim 5, wherein the pyrolysis is not carried out with addition of hydrogen compounds.

7. The process according to claim 5, wherein the pyrolysis is carried out with addition of hydrogen (hydrocracking).

8. The process according to any of claims 5 to 7, wherein the pyrolysis is carried out using a black liquor material which under normal conditions (20° C., 1013 mbar) has a liquid content of not more than 70% by weight, preferably not more than 50% by weight, based on the total weight of the black liquor material.

9. The process according to any of claims 1 to 4, wherein, in step b), the biomass starting material provided is subjected to a decomposition in the liquid phase.

10. The process according to claim 9, wherein, in step b), the biomass starting material, preferably a lignin-comprising starting material, is subjected to a decomposition in the presence of an aqueous-alkaline, aqueous-acidic or organic decomposition medium.

11. The process according to either claim 9 or 10, wherein the decomposition is carried out using at least one cellulose-depleted fraction from a pulp process, in particular a black liquor from the kraft process.

12. The process according to any of the preceding claims, wherein, in step c), the separation into at least one aromatics-enriched fraction C1) and at least one aromatics-depleted fraction C2) is effected by distillation, extraction, absorption, a membrane process or a combination thereof, preferably by distillation, extraction, absorption or a combination thereof.

13. The process according to any of claims 5 to 8, wherein, in step b), the biomass starting material provided in step a) is subjected to a pyrolysis to effect decomposition and, in step c), the separation into at least one aromatics-enriched fraction C1) and at least one aromatics-depleted fraction C2) comprises an absorption.

14. The process according to any of claims 9 to 11, wherein, in step b), the biomass starting material provided in step a) is subjected to a decomposition in the liquid phase and, in step c), the separation into at least one aromatics-enriched fraction C1) and at least one aromatics-depleted fraction C2) comprises an extraction and/or a distillation.

15. The process according to claim 14, wherein the separation into at least one aromatics-enriched fraction C1) and at least one aromatics-depleted fraction C2) in step c) comprises the following substeps:
c1) extraction of the decomposition product obtained in step b) to give an aromatics-enriched extract and an aromatics-depleted residue,
c2) optionally separation of the extract into an extractant-enriched and aromatics-depleted fraction and an aromatics-enriched and extractant-depleted fraction,
c3) introduction of the aromatics-enriched extract obtained in step c1) or the aromatics-enriched fraction obtained in step c2) into step d).

16. The process according to any of the preceding claims, wherein the decomposition product obtained in step b) or the fraction C1) isolated therefrom in step c) has predominantly components having a molecular weight of not more than 500 g/mol.

17. The process according to any of the preceding claims, wherein the aromatics-depleted fraction C2) isolated in step c) is at least partly used for producing synthesis gas.

18. The process according to any of the preceding claims, wherein the reaction in step d) comprises a hydrodealkylation or a steam dealkylation or a mixed form derived therefrom.

19. The process according to any of the preceding claims, wherein the temperature in the dealkylation zone is in the range from 400 to 900° C., preferably from 500 to 800° C.

20. The process according to any of the preceding claims, wherein the absolute pressure in the dealkylation zone is in the range from 1 to 100 bar, particularly preferably from 1 to 20 bar, in particular from 1 to 10 bar.

21. The process according to any of the preceding claims, wherein the discharge from the dealkylation zone in step e) is subjected to a separation to give the following three streams:
E1) a stream enriched in single-ring aromatics which are unalkylated or have a low degree of alkylation,
E2) a stream enriched in aromatics which are not dealkylated or have a low degree of dealkylation,
E3) a stream enriched in by-products which are more volatile than E1) and E2).

22. The process according to any of the preceding claims, wherein the separation of the discharge from the dealkylation zone in step e) comprises an absorption.

23. The process according to claim 22, wherein the separation of the discharge from the dealkylation zone in step e) comprises the following substeps:
e1) contacting of the discharge from the dealkylation zone with an absorption medium to give an absorbate enriched in aromatic primary products from the dealkylation and a gas stream E3) depleted in aromatic primary products from the dealkylation,
e2) separation of the absorbate into a stream E1) enriched in single-ring aromatics which are unalkylated or have a low degree of alkylation, a stream E2) which is enriched in aromatics which are not dealkylated or have a low degree of dealkylation and optionally a stream comprising the absorption medium,
e3) recycling if present, of the stream comprising the absorption medium to step e1),
e4) optionally recycling of at least part of the stream E2) to the dealkylation zone of step d).

24. The process according to any of claims 21 to 23, wherein the stream E3) obtained in step e) is at least partly used for producing synthesis gas.

25. The process according to any of the preceding claims, wherein a synthesis gas-comprising stream or a hydrogen-enriched stream produced from the synthesis gas is fed into the decomposition in step b) and/or into the dealkylation in step d).
